(19) United States
(12) Patent Application Publication
(10) Pub. No.: US 2002/0183684 A1
(43) Pub. Date: Dec. 5, 2002

(54) Electroporation-enhanced inhibition of vascular neointimal hyperplasia

(75) Inventors: Nagendu B. Dev, San Diego, CA (US); Gunter A. Hofmann, San Diego, CA (US); Sukhendu B. Dev, San Diego, CA (US); Dietmar P. Rabussay, Solana Beach, CA (US)

Correspondence Address: Gray Cary Ware & Freidenrich LLP, Suite 1100, 4365 Executive Drive, San Diego, CA, 92121-2133, US

(73) Assignee: GENETRONICS

(21) Appl. No.: 10/171,261

(22) Filed: Jun. 12, 2002

Related U.S. Application Data
(63) Continuation of application No. 09/737,716, filed on Dec. 15, 2000, now abandoned.
(63) Continuation of application No. 09/329,098, filed on Jun. 9, 1999.
Application No. 09/329,098 is a division of application No. 08/668,725, filed on Jun. 24, 1996, now Pat. No. 5,944,710.
(60) Provisional application No. 60/171,006, filed on Dec. 15, 1999.

(51) Int. Cl. A61N 1/30

(52) U.S. Cl. 604/20; 604/96.01

(57) ABSTRACT

The present invention provides methods for inhibiting or preventing hyperplastic intimal growth by intravascular administration of a composition comprising heparin, or a derivative thereof in conjunction with at least one electric pulse having sufficient strength and duration to cause electroporation of the cells lining the blood vessel. Such treatment inhibits or prevents hyperplastic intimal growth in vessels, such as arteries, as compared with a non-electroporated vessel to which the heparin, or derivative thereof, is administered. In another aspect, the present invention provides methods for electroporation-enhanced local delivery of heparin to cells lining an artery in a subject by directly applying at least one electric pulse to the interior surface of the artery in conjunction with local application of a composition comprising heparin, or a derivative thereof, said electric pulse having sufficient strength and duration to locally deliver the heparin to the artery so as to decrease hyperplastic intimal growth compared with that in an untreated region of the artery. A unique intravascular porous balloon electroporation catheter can be used to apply the composition directly to the arterial wall.

### RELATED APPLICATION

This application relies for priority under 35 U.S.C. §119(e)(1) on provisional application Serial No. 60/171,006, filed Dec. 15, 1999 and is a Continuation-In-Part application of U.S. patent application Ser. No. 09/329,098, filed Jun. 9, 1999, now pending, which is a divisional application of U.S. application Ser. No. 08/668,725, filed Jun. 24, 1996, now issued as U.S. Pat. No. 5,944,710.

### FIELD OF THE INVENTION

The present invention generally relates to methods for enhancing the effectiveness of methods of drug delivery using electroporation. In particular, the present invention relates to use of electroporation-enhanced inhibition of vascular neointimal hyperplasia.

### BACKGROUND OF THE INVENTION

For some time now, it has been known that electric fields could be used to create pores in cells without causing permanent damage to them. This discovery made possible the insertion of large molecules into cell cytoplasm. It is known that genes and other molecules such as pharmacological compounds can be incorporated into live cells through a process known as electroporation.

Treatment of cells by electroporation is carried out by infusing a composition into a patient and applying an electric field to the desired site of treatment between a pair of electrodes. The field strength must be adjusted reasonably accurately so that electroporation of the cells occurs without damage, or at least minimal damage, to any normal or healthy cells. The distance between the electrodes can then be measured and a suitable voltage according to the formula E=V/d can then be applied to the electrodes (E=electric field strength in V/cm; V=voltage in volts; and d=distance in cm).

Studies have also shown that large size nucleotide sequences (up to 630 kb) can be introduced into mammalian cells via electroporation (Eanault, et al., Gene (Amsterdam), 144(2):205, 1994; Nucleic Acids Research, 15(3):1311, 1987; Knutson, et al., Anal. Biochem., 164:44, 1987; Gibson, et al., EMBO J., 6(8):2457, 1987; Dower, et al., Genetic Engineering, 12:275, 1990; Mozo, et al., Plant Molecular Biology, 16:917, 1991), thereby affording an efficient method of gene therapy, for example.

Iontophoresis uses electrical current to activate and to modulate the diffusion of a charged molecule across a biological membrane, such as a cell membrane, in a manner similar to passive diffusion under a concentration gradient, but at a facilitated rate. In general, iontophoresis technology uses an electrical potential or current across a semipermeable barrier. Delivery of heparin molecules to patients has been shown using iontophoresis (IO), a technique which uses low current (d.c.) to drive charged species into the arterial wall. lontophoretic delivery of heparin (1000 U/ml) into porcine artery was shown to be safe and well tolerated without any change in the coronary angiography or normal physiological parameters such as blood pressure and cardiac rhythm. Although heparin in varying concentration from 1000 U to 20,000 U/ml results in greater concentrations remaining in the vessel after IO delivery compared to passive delivery, approximately 1 hour after the delivery of heparin, 96% of the drug washes out (Mitchel, et al., ACC 44th Annual Scientific Session, Abs.#092684, 1994). It has also been reported that platelet deposition following IO delivery of heparin is reduced in the pig balloon injury model. ¹²⁵I-labeled hirudin has also been delivered iontophoretically into porcine carotid artery (Fernandez-Ortiz, et al., Circulation, 89:1518, 1994). A local concentration of hirudin can be achieved by IO; however, as with the above experiments with heparin, 80% of the drug washes out in 1 hour and after three hours, the level is the same as for passive delivery.

Heparins are widely used therapeutically to prevent and treat venous thrombosis. Apart from interactions with plasma components such as antithrombin III or heparin cofactor II, interactions with blood and vascular wall cells may underlie their therapeutic action. The term heparin encompasses to a family of unbranched polysaccharide species consisting of alternating 1→4 linked residues of uronic acid (L-iduronic or D-glucuronic) and D-glucosamine. Crude heparin fractions commonly prepared from bovine and porcine sources are heterogeneous in size (3,000-40,000 daltons), monosaccharide sequence, sulfate position, and anticoagulant activity. Mammalian heparin is synthesized by connective tissue mast cells and stored in granules that can be released to the extracellular space following activation of these cells. Overall, heparin is less abundant than related sulfated polysaccharides, such as heparin sulfate, dermatan sulfate, and chondroitin sulfate, which are synthesized in nearly all tissues of vertebrates. Heparin and these other structures are commonly referred to as glycosaminoglycans.

The anticoagulant activity of heparin derives primarily from a specific pentasaccharide sequence present in about one third of commercial heparin chains purified from porcine intestinal mucosa. This pentasaccharide, αG1cNR16Sβ(1-4)G1cA. (1-4)G1cNS3S6R2α(1-4)IdoA2Sα(1-4)G1cNS6S, where R1=—SO₃— or —COCH₃ and R2=—H or —SO₃—, is a high affinity ligand for the circulating plasma protein, antithrombin (antithrombin III, AT-III), and upon binding induces a conformational change that results in significant enhancement of antithrombin's ability to bind and inactivate coagulation factors, thrombin, Xa, IXa, VIIa, XIa and XIIa. For heparin to promote antithrombin's activity against thrombin, it must contain the specifically recognized pentasaccharide and be at least 18 saccharide units in length. This additional length is believed to be necessary in order to bridge antithrombin and thrombin, thereby optimizing their interaction. Other polymers found in heparin have platelet inhibitory effects or fibrinolytic effects. In clinical development are the low molecular weight heparins (LMW), which contain only the specific polymers required for antithrombin III activation. These low molecular weight derivatives have greater specific antithrombotic activity and less antiplatelet activity. They also have the characteristic of being easier to dose and being safer.

A major objective of many biotechnology companies and pharmaceutical industries is to find safe, easy and effective ways of delivering drugs and genes into the arterial wall by a variety of means. Brief reviews have appeared on gene transfer methods related to cardiology (Dzau, et al., TIBTECH, 11:205, 1993; Nabel, et al., TCM, Jan.-Feb, issue: 12, 1991). Retroviruses, despite their high efficiency of transfer, have various limitations, such as 1) size (<8 kb), 2) potential for activation of oncogenes, 3) random integration and, 4) inability to transfect non-dividing cells. Other viral vectors such as adenovirus are efficient but have the potential risk of infection and inflammation. HVJ-mediated transfection, although highly efficient, can exhibit non-specific binding. Liposomes, which have become very popular, are safe and easy to work with, but have low efficiency and long incubation times. Recent changes in the formulation of liposomes have, however, has increased their efficiency several fold.

Catheter delivery systems, with many different balloon configurations, have also been used to locally deliver genes and/or drugs. These include: hydrogel balloon, laser-perforated (Wolinsky balloon), ‘weeping,’ channel and ‘Dispatch’ balloons and variations thereof (Azrin, et al., Circulation, 90:433, 1994; Consigny, et al., J Vasc. Interv. Radiol., 5:553, 1994; Wolinsky, et al., JACC, 17:174B, 1991; Riessen, et al., JACC, 23:1234, 1994; Schwartz, Restenosis Summit VII, Cleveland, Ohio, 1995, pp 290-294). Delivery capacity with hydrogel balloon is limited and, during placement, the catheter can lose substantial amount of the drug or agent to be introduced. High pressure jet effect in Wolinsky balloon can cause vessel injury which can be avoided by making many holes, <1 μm, (weeping type). The ‘Dispatch’ catheter has generated a great deal of interest for drug delivery and it create circular channels and can be used as a perfusion device allowing continuous blood flow.

Extensive research efforts have been expended in search of effective technologies or drug therapies for the treatment of inflammatory proliferative diseases such as restenosis after percutaneous transluminal angioplasty (PTA) or endoluminal stenting (ELS) procedures. Clinical trials of drugs directed towards different mechanisms suspected to be contributing to the restenotic process, e.g., platelet aggregation, inflammation and cell proliferation, have proven unsuccessful in reducing the restenosis rate (D. Brieger and E. Topol, Cardiovasc Res 35(3):405-413, 1997). Drugs tested included antiplatelet agents, anticoagulants, corticosteroids, calcium channel blocking agents and colchicine (E. Camenzind, et al,. Semin Interv Cardiol 1(1):67-76, 1996). These drugs were either delivered systemically by injection or infusion, or locally by a variety of drug delivery catheters. Intravascular delivery of genes by viral vectors (P. D. Kessler et al. Proc Natl Acad Sci 93:14082-14087, 1996) or lipofection (J. G. Pickering et al., Circulation 89:13-21, 1994) has also been attempted for vascular therapy without significant success.

Costly procedures like ELS combined with either γ or β radiation (SCRIPPS and BERT studies, respectively) (D. O. Williams, Am J Cardiol 81(7A): 18E-20E, 1998) have met with limited success in preventing restenosis. Their full long-term effects are unknown but radiation effects have been associated with newly formed vascular lesions (R. Virmani et al., Semin Interv Cardiol 3(3-4):163-172, 1998).

One of the central events in the restenotic process is an abnormal proliferation and migration of vascular smooth muscle cells from the media into the intima, in response to a variety of growth factors and inflammatory cytokines, resulting in neointimal hyperplasia (J. J. Castronuovo et al., Cardiovasc Surg 3(5):463-468 1995). Heparin is known to possess an inhibitory effect on smooth muscle cell (SMC) proliferation (A. W. Clowes and M. M. Clowes, Circ Res 58:839-845, 1986) and its inhibitory effect on migration of smooth muscle cells has been verified in cell culture systems involving both rat and bovine smooth muscle cell experimental models (A. Chajara et al., J Cardiovasc Pharmacol 23:995-1003, 1994). The limited success of pharmacological agents, such as heparin, in obtaining a long-term therapeutically desirable effect in limiting proliferation could be partially attributed to insufficient intramural drug levels in the target lesion. Current drug delivery technologies (e.g., porous balloon or double balloon catheters, alone or in combination with iontophoresis, ultrasound or other auxiliary methods) are limited by a time span of approximately one minute during which blood flow can be temporarily interrupted to achieve high local drug concentrations in the vessel segment to be treated. This time span is apparently insufficient to allow adequate vessel penetration by the therapeutic agents. In addition, present delivery methods result at best in interstitial, but not intracellular drug delivery. Thus, once blood flow is restored, a significant fraction of the drug is washed out of the target arterial segment, resulting in a reduction of the vascular concentration of the drug (R. L. Wilensky et al., Am Heart J 129:852-859, 1995) and, consequently, in little or no therapeutic efficacy.

It has been shown that the normal membrane permeability barrier of eukaryotic cells can be transiently breached by subjecting the cells to brief, high-intensity electrical fields (U. Zimmerman, Biochim Biophys Acta 694:227-277, 1982; G. A. Hofmann and G. A. Evans, IEEE Eng Med Biol 5:6-25, 1986), thereby facilitating the entry of macromolecules and even of microparticles during this electroporation or electropermeabilization process (I. Hapala, Crit Rev Biotech 17:105-122, 1997). The efficiency of electroporation in delivering low and high molecular weight drugs ex vivo and in vivo has been shown in numerous examples including the loading of platelets with the prostacyclin analog, iloprost (N. Crawford and N. Chronos, Semin Interv Cardiol 1:91-102 (1996)) and the delivery of heparin into arterial walls (N. B. Dev et al. Cathet Cardiovasc Diagn 45:337-345, 1998).

Due to its short time requirement (<1 min), electroporation has the advantage of minimal disturbance of blood flow, with the therapeutic agent being delivered into the interstitial space as well as into the cells of the vessel wall. Thus, a smaller fraction of the drug is lost to the washout effect (N. B. Dev et al., Cathet Cardiovasc Diagn 45:337-345, 1998). High local drug concentrations achieved by electroporation may prove sufficiently effective for the treatment of vascular disorders without the adverse side effects seen in other treatment modalities requiring high systemic drug levels, e.g., thrombocytopenia induced by high levels of heparin (L. C. Wang et al., Eur J Clin Invest 29(3):232-237 (1999)).

It has previously been shown that an enhancement in uptake and retention of fluorescent-tagged heparin inside the rabbit arterial wall in vivo can be achieved using an electroporation catheter (N. B. Dev et al., Cathet Cardiovasc Diagn 45:337-345, 1998). However, this study failed to show whether the transferred heparin would maintain its functional activity and whether the achieved distribution, intracellular concentration and residence time of the heparin in the arterial wall would be sufficient to result in a therapeutic effect.

Thus, there is a need in the art for new and better methods for inhibiting and preventing neointimal growth in blood vessels, such as those which have been damaged by trauma, harsh chemicals, and the like. Especially, there is a need in the art for new and better methods for inhibiting restenosis following balloon angioplasty in which the partially occluded blood vessel is stretched to counteract the effects of arterial narrowing caused by plaque build-up, or neointimal growth.

### BRIEF SUMMARY OF THE INVENTION

The present invention overcomes these and other problems in the art by providing methods for inhibiting or preventing hyperplastic intimal growth in a blood vessel in a subject by administering a composition comprising heparin or a derivative thereof locally to the vessel in the subject and applying at least one electric pulse directly to cells lining the vessel. The at least one electric pulse has sufficient strength and duration to cause electroporation of the cells, thereby delivering the composition into the cells so as to prevent or inhibit local hyperplastic intimal growth in the vessel wall as compared with a vessel having non-electroporated cells to which the composition is administered.

In another embodiment according to the present invention, there are provided methods for electroporation-enhanced local delivery of heparin to cells lining a blood vessel in a subject in need thereof by administering a composition comprising heparin or a derivative thereof locally to the vessel in the subject and applying at least one electric pulse directly to cells lining the vessel. The at least one electric pulse has sufficient strength and duration to cause electroporation of the cells, thereby delivering the composition into the cells so as to decrease local hyperplastic intimal growth, as compared with an untreated vessel.

### BRIEF DESCRIPTION OF THE DRAWINGS

FIG. 1 is a schematic illustration of an endoluminal catheter.

FIG. 2A-B, a computer images of fluoresceinated heparin in the pulsed rabbit artery (FIG. 2A), and in the non-pulsed artery (FIG. 2B).

FIG. 3A-D shows confocal microscopy images of rabbit arteries after fluoresceinated heparin treatment. R1L1 shows the left artery, no pulse; R1R1 shows the right artery, with pulse; R2L1 shows the left artery, with pulse; and R2E1 shows the right artery, no pulse.

FIG. 4A-D shows confocal microscopy fluorescent images of rabbit arteries after heparin treatment. 4L2 shows left artery with pulse; 4R2 shows right artery no pulse; 4L1 shows left artery with pulse; and 1L3 shows left artery no pulse.

FIG. 5A and 5B shows confocal microscopy fluorescent images of rabbit arteries after heparin treatment. 12R1, right artery with pulse and 12L1, left artery, no pulse.

FIG. 6 is a schematic diagram of a rabbit treated by the method of the invention, including the catheter description.

FIG. 7 is a schematic diagram of an exemplary endoluminal electroporation catheter of the invention.

FIGS. 8A-C, show x-rays of insertion of the catheter into the carotid artery (FIG. 8A), infusion of radiocontrast dye (FIG. 8B), and balloon inflation (FIG. 8C), respectively.

FIG. 9 is a schematic diagram representing of an electroporation-assisted heparin delivery system (Genetronics, Inc.) inserted within an arterial section. The system comprises a porous balloon electroporation catheter (PBE) wherein the balloon serves as a reservoir for heparin, and which is electrically connected to a T-820 square voltage wave generator.

FIGS. 10A and 10B are bar graphs showing morphometric measurements 28 days post injury of untreated, non-injured rat arteries (Group 3) and of balloon injured rat arteries (treated with heparin alone (Group 1) or heparin and electroporation (Group 3)). FIG. 10A shows the intima/media ratio (R) in the three groups; FIG. 10B shows active lumen area (mm sq) in the three groups. H=Heparin; E=Electroporation; Inj=Balloon injury. (+) and (−) indicate presence or absence, respectively, of H, E or Inj.

FIG. 11 is a graph showing the external elastic lamina area (EEL in mm²) (ordinate) as a function of internal elastic lamina area (IEL) for balloon-injured and non-injured rat arteries after local delivery of heparin in the absence or presence of pulsed electric fields.

+=H−E− Inj+; Δ=H+E+ Inj+; o=H−E− Inj−.

FIG. 12 is a graph showing the influence of vessel cross-section on lumen dimension. Cross sectional morphometry of perfusion-fixed injured and non-injured carotid arteries demonstrates correlation of lumen area with artery size. The remodeling capacity of the heparinized, electroporated arteries (H+E+) is shown to be better than that of the heparinized, non-electroporated arteries (H+E−). Inj (+) or (−) indicates, respectively, arteries injured by balloon expansion or non-injured arteries.

### DETAILED DESCRIPTION OF THE INVENTION

The present invention provides methods for the local, controlled, and sustained intravascular delivery of a therapeutic composition to a vessel in a subject using electroporation techniques. The methods utilize pulsed electric fields and has an advantage of allowing lower concentrations of compositions to be utilized as opposed to high dosages typically used with passive delivery modalities.

In one embodiment according to the present invention, there are provided methods for inhibiting or preventing hyperplastic intimal growth in a blood vessel in a subject by administering a composition comprising heparin or a derivative thereof locally to the vessel in the subject and applying at least one electric pulse directly to cells lining the vessel. The at least one electric pulse has sufficient strength and duration to cause electroporation of the cells, thereby delivering the composition into the cells so as to prevent or inhibit local hyperplastic intimal growth in the vessel wall as compared with a vessel having non-electroporated cells to which the composition is administered.

In another embodiment according to the present invention, there are provided methods for electroporation-enhanced local delivery of heparin to cells lining a blood vessel in a subject in need thereof by administering a composition comprising heparin or a derivative thereof locally to the vessel in the subject and applying at least one electric pulse directly to cells lining the vessel. The at least one electric pulse has sufficient strength and duration to cause electroporation of the cells, thereby delivering the composition into the cells so as to decrease local hyperplastic intimal growth, as compared with an untreated vessel.

In yet another embodiment according to the present invention there are provided delivery systems that allows controlled sustained, high local concentrations of pharmacologic agents to be delivered directly at a site without exposing the entire circulation to the agent. Pharmacologic approaches to inhibit smooth muscle cells migration and proliferation, for example, have been effectively used at supraphysiological doses in animal research studies. However, such high concentrations may be impractical for clinical use in humans because of the risk of systemic side effects and the lack of specific targeting of drugs given systemically at such high dosages. This invention is clinically relevant for the local treatment of arteries undergoing catheter-based interventions, such as angioplasty, atherectomy, rotablating or stenting, for example.

In various embodiments, the invention provides various methods for sustained intravascular delivery of a composition to a subject. The methods include administering the composition to the subject and applying an electrical impulse to a vessel via electroporation, wherein the impulse is of sufficient strength and time for the impulse to cause electroporation of at least one cell in the interior of the vessel such that the composition is delivered into the cells in the vessel and is retained in the vessel thereby resulting in sustained delivery. In one aspect of the invention, iontophoresis can be employed to further deliver the composition to a cell, either prior to, simultaneously with or after electroporation.

The term “sustained” as used herein means that once the composition is delivered to the vessel, it is retained in the vessel for a period of time of as long as 24 to about 36 hours, and typically for 12 hours. In other words, there is no appreciable washout of the composition as compared with the concentration of the composition delivered under conventional delivery (e.g., passive diffusion or IO).

The terms “intravascular” and “vessel” mean any artery, vein or other “lumen” in the subject's body to which the electric pulse can be applied and to which the composition can be delivered. A lumen is known in the art as a channel within a tube or tubular organ. Examples of preferred vessels in the methods of the invention include the coronary artery, carotid artery, the femoral artery, and the iliac artery. While not wanting to be bound by a particular theory, it is believed that the electric impulse applied to the vessel allows the delivery of the composition primarily to the cells of the medial region of the vessel, but also to the intima and less so to the adventitia.

The composition delivered by the methods of the invention includes any composition which would have a desired biological effect at the site of electroporation. For example, preferred compositions include antithrombotic, antirestenoitic, antiplatelet, and antiproliferative compositions, especially heparin-containing compositions. Other compositions include platelet receptor and mediator inhibitors, smooth muscle cell proliferation inhibitors, growth factor inhibitors, GpIlb/IIa antagonists, agents that inhibit cell adhesion and aggregation, agents that block thromboxane receptors, agents that block the fibrinogen receptor, etc. Specific examples of such compositions include heparin (including high (e.g., having a molecular weight greater than about 18,000) and low molecular weight (e.g., having a molecular weight of about 2,500 to about 18,000) and fragments thereof), hirulog, tissue plasminogen activator (tPA), urokinase, streptokinase, warfarin, hirudin, angiotensin converting enzyme (ACE) inhibitors, PDGF-antibodies, proteases such as elastase and collagenase, serotonin, prostaglandins, vasoconstrictors, vasodialators, angiogenesis factors, Factor VIII or Factor IX, TNF, tissue factor, VLA-4, growth-arrest homeobox gene, gax, L-arginine, GR32191, sulotroban, ketanserin, fish oil, enoxaprin, cilazapril, forinopril, lovastatin, angiopeptin, cyclosporin A, steroids, trapidil, colchicine, DMSO, retinoids, thrombin inhibitors, antibodies to von Willebrand factor, antibodies to glycoprotein IIb/IIIa, calcium chelation agents, etc. Other therapeutic agents (e.g., those used in gene therapy, chemotherapeutic agents, nucleic acids (e.g., polynucleotides including antisense, for example c-myc and c-myb), peptides and polypeptides, including antibodies) may also be administered by the methods of the invention.

The therapeutic composition can be administered alone or in combination with each other or with another agent. Such agents include combinations of tPA, urokinase, prourokinase, and streptokinase, for example. Administration of heparin with tissue plasminogen activator would reduce the dose of tissue plasminogen activator that would be required, thereby reducing the risk of clot formation which is often associated with the conclusion of tissue plasminogen activator and other thrombolytic or fibrinolytic therapies.

Compositions used in the various methods of the invention include biologically functional analogues of the compositions described herein. For example, such modifications include addition or removal of sulfate groups, addition of phosphate groups and addition of hydrophobic groups such as aliphatic or aromatic aglycones. Modifications of heparin, for example, include the addition of non-heparin saccharide residues such as sialic acid, galactose, fucose, glucose, and xylose. When heparin is used as the composition, it may include a fragment of naturally occurring heparin or heparin-like molecule such as heparan sulfate or other glycosaminoglycans, or may be synthetic fragments. The synthetic fragments could be modified in saccharide linkage in order to produce more effective blockers of selectin binding. Methods for producing such saccharides will be known by those of skill in the art (see for example: M. Petitou, Chemical Synthesis of Heparin, in Heparin, Chemical and Biological Properties, Clinical Applications, 1989, CRC Press Boca Raton, Fla. D. A. Lane and V. Lindahl, eds. pp. 65-79).

The composition administered by the methods of the invention may be a mixture of one or more compositions, e.g., heparin and tPA. Further, compositions such as heparin may include a mixture of molecules containing from about 2 to about 50 saccharide units or may be homogeneous fragments as long as the number of saccharide units is 2 or more, but not greater than about 50.

Where a disorder is associated with the expression of a gene (e.g., IGF-1, endothelial cell growth factor), nucleic acid sequences that interfere with the gene's expression at the translational level can be delivered. This approach utilizes, for example, antisense nucleic acid, ribozymes, or triplex agents to block transcription or translation of a specific mRNA, either by masking that mRNA with an antisense nucleic acid or triplex agent, or by cleaving it with a ribozyme.

Preferably the subject is a human, however, it is envisioned that the methods of sustained in vivo delivery of compositions via electroporation as described herein can be performed on any animal.

Preferably, the therapeutic composition is administered either prior to or substantially contemporaneously with the electroporation treatment. The term “substantially contemporaneously” means that the therapeutic composition and the electroporation treatment are administered reasonably close together with respect to time. The chemical composition of the agent will dictate the most appropriate time to administer the agent in relation to the administration of the electric pulse. The composition can be administered at any interval, depending upon such factors, for example, as the nature of the clinical situation, the condition of the patient, the size and chemical characteristics of the composition and half-life of the composition.

The composition administered in the methods of the invention can be administered parenterally by injection or by gradual perfusion over time, for example over a period of about 5 to about 50 seconds. The composition can be administered intravenously, intraperitoneally, intramuscularly, subcutaneously, intracavity, or transdermally, and preferably is administered intravascularly at or near the site of electroporation.

Preparations for administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's , or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Further, vasoconstrictor agents can be used to keep the therapeutic composition localized prior to pulsing.

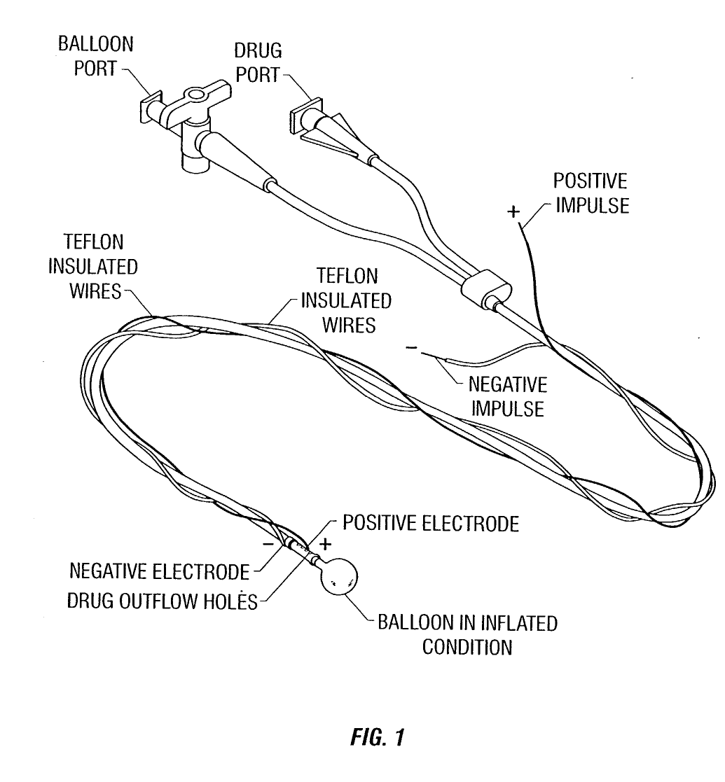

In another embodiment, the invention provides a catheter device 100 useful in the methods of the invention that can be modified as described herein, as shown in FIGS. 1, 6, and 7. The catheter may be, for example, a modified Berman catheter (Arrow International, Inc., Reading, Pa.). One of skill in the art will know of other balloon catheter devices for endoluminal electroporation mediated drug delivery that can be modified according to the present invention.

The catheter 100 may include at least one inflatable balloon 102 near the distal end of the catheter 100, and at least one inflation port 104 for inflating each balloon 102, in a conventional manner. The catheter 100 also includes a first electrode 110 and a second electrode 112 that are coupled by wires to a voltage source generator 114, which may be, for example, an ECM 600 exponential generator from BTX, a division of Genetronics, Inc., San Diego, Calif. The first electrode 110 is preferably placed close to at least one infusion opening 120. In one embodiment, the infusion openings 120 may be coincident with the first electrode 110, such that the first electrode 110 completely surrounds at least one infusion opening 120.

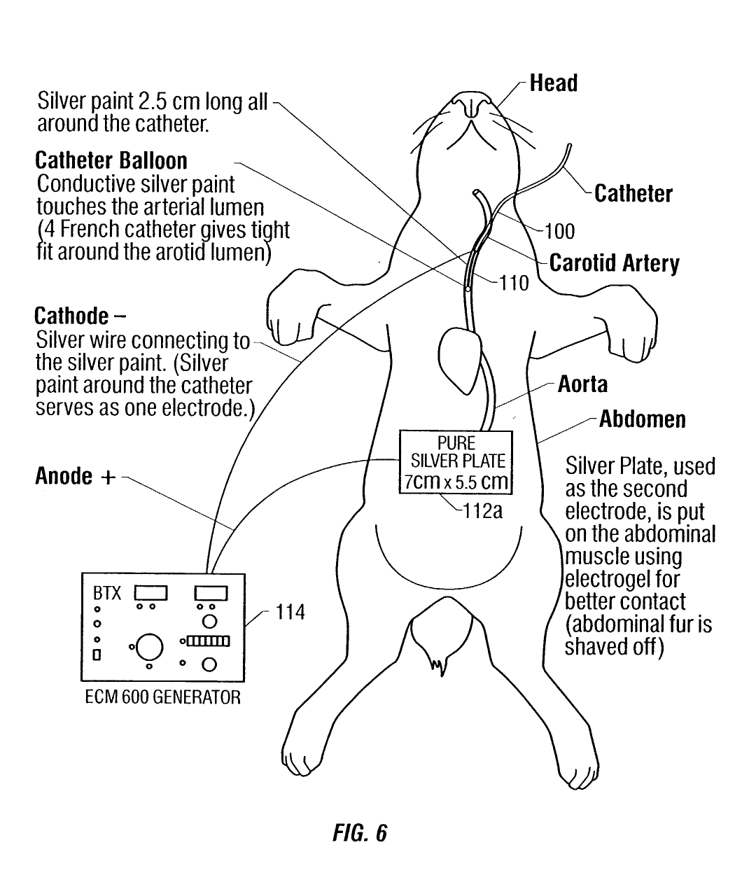

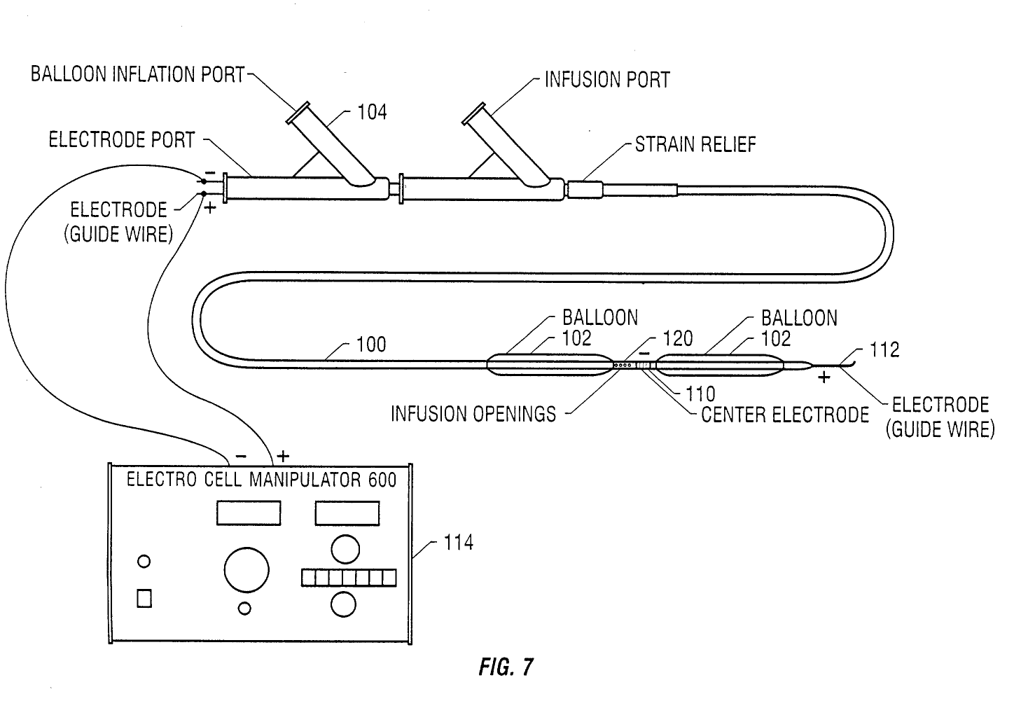

The first electrode 110 is preferably made of an electrically conductive material that is biologically compatible, e.g., biologically inert, with a subject. Examples of such material include silver or platinum wire wrapped around or laid on or near the surface of the catheter 100; a plated or painted coating of conductive material, such as silver paint, on some portion of the catheter 100; or a region of the catheter 100 that has been made conductive by implantation (during or after manufacture, such as by ion implantation) of electrically conductive materials, such as powdered metal or conductive fibers. The conductor need not be limited to metal, but can be a semiconductor or conductive plastic or ceramic. For ease of manufacture, the embodiments illustrated in FIGS. 6 and 7 use conductive silver paint for the first electrode 110 as a coating on approximately 2.5 cm of the length of the catheter 100 near the infusion ports 120.

The second electrode 112 similarly comprises an electrically conductive material, and can be of the same or different type of conductive material as the first electrode 110. In the embodiment shown in FIG. 6, the second electrode comprises a silver plate 112a configured to be applied to a portion of the body of a subject such that an electric field sufficient to cause electroporation of at least one cell in a vessel is generated when voltage from the voltage source 114 is applied to the first electrode 110 and the second electrode 112. The second electrode, when placed externally, is preferably placed on bare skin (e.g., shaved abdominal muscle of the subject), preferably using a conductive gel for better contact. FIG. 7 shows that the second electrode 112 may be a conductive guide wire for the catheter 100.

The first electrode 110 and the second electrode 112 are coupled to the voltage source 114 by conductors, which may be, for example, silver or platinum wires, but can be any conductive structure, such as flexible conductive ink within the catheter 100 for connecting the first electrode 110.

The infusion ports 120 can be made during or after manufacture of the catheter 100, and can be placed on one or both sides of the first electrode 110, or within the bounds of the first electrode 110.

In an alternative embodiment, the second electrode 112 may be formed in a manner similar to the first electrode 110 and positioned between the first electrode 110 and the infusion openings 120, or positioned with the infusion openings 120 between the first electrode 110 and the second electrode 112. Other configurations of the first electrode 110 and the second electrode 112 can be utilized, such as interdigitated electrodes with infusion openings 120 nearby or between the interdigitated “fingers” of the electrodes, or as concentric rings with the infusion openings within the centermost ring, between the centermost and outermost ring, and/or outside of the outermost ring. Additional configurations are within the scope of the present invention so long as they provide a structure that, when supplied by voltage from the voltage source 114, generates an electric field sufficient to cause electroporation of at least one cell in the vessel.

In operation, the catheter 100 is positioned so that a balloon 102 traverses or crosses a stenotic lesion, for example, and the balloon 102 is inflated to expand the vessel (e.g., an artery or vein), thereby dilating the lumen of the vessel. Preferably the ballon is expanded to have an exterior diameter about 20% greater than the resting vessel lumen diameter during infusion. A therapeutic composition is delivered into the vessel via the infusion openings 120, and at least during part of the time before, during, or after infusion occurs, electrical pulses from the voltage source 114 are applied to the first electrode 110 and second electrode 112 so as to cause electroporation of at least one cell in the vessel. Following delivery of the therapeutic composition to such cell, the catheter may be withdrawn, unless additional composition delivery and electroporation is desired.

The methods described above are also applicable with metallic stents. The stent itself forms one set of electrodes while a guide wire acts as the second electrode. Stents, on their own, or coated with heparin, are useful for reduction of restenosis. Such results can be further augmented when combined with pulsed electric fields. This would be particularly suitable for angioplasty where a stent is deployed. (For detailed review, see de Jaegere, P. P. et al., Restenosis Summit Proc. VIII, 1996, pp 82-109). Stent implantation, along with local delivery of antirestenotic drugs, such as heparin, by pulsed electric fields reduces the restenosis rate. Besides a normal stent, a retractable or biodegradable stent can also be used with this mode of delivery.

In another aspect of the invention, the described methods are useful for bypass grafts. These can include aortocoronary, aortoiliac, aortorenal, femoropopliteal. In the case of a graft with autologous or heterologous tissue, the cells in the tissue can be electroporated, ex vivo, with a nucleic acid encoding a protein of interest. Since electroporation is relatively fast, a desired nucleic acid can be transferred in a saphenous vein, e.g., outside the body, while the extracorporeal circulation in the patient is maintained by a heart-lung machine, and the vein subsequently grafted by standard methods. Where synthetic material is used as a graft, it can serve as a scaffolding where appropriate cells containing a nucleic acid sequence of interest that has been electroporated, ex vivo, can be seeded.

The methods of the invention can be used to treat disorders by delivery of any composition, e.g., drug or gene, with a catheter, as described herein. For example, patients with peripheral arterial disease, e.g., critical limb ischemia (Isner, J. M. et al, Restenosis SummitVIII, Cleveland, Ohio, 1996, pp 208-289) can be treated as described herein. Both viral and non-viral means of gene delivery can be achieved using the methods of the invention. These include delivery of naked DNA, DNA-liposome complex, ultraviolet inactivated HVJ (haematoagglutanating virus of Japan) liposome vector, delivery by particle gun (e.g., biolistics) where the DNA is coated to inert beads, etc. Various nucleic acid sequences encoding a protein of interest can be used for treatment of cardiovascular disorders, for example. The expression of the growth factors PDGF-B, FGF-1 and TGF β1 has been associated with intimal hyperplasia, therefore, it may be desirable to either elevate (deliver sense constructs) or decrease (deliver antisense) such gene expression. For example, whereas PDGF-B is associated with smooth muscle cell (SMC) proliferation and migration, FGF-1 stimulates angiogenesis and TGF β1 accelerates procollagen synthesis.

Any composition that inhibits SMC proliferation and migration, platelet aggregation and extracellular modeling is also desirable for use in the electroporation-mediated delivery methods of the invention. Such compositions include interferon-γ. which inhibits proliferation and expression of α-smooth muscle actin in arterial SMCs and non-protein mediators such as prostaglandin of the E series.

Examples of other genes to be delivered by the methods of the invention includes Vascular endothelial growth factor (VEGF) and endothelial specific mitogen, which can stimulate angiogenesis and regulate both physiologic and pathologic angiogenesis.

Administration of the composition in the various methods of the invention may be used for ameliorating conditions caused by various types of injury (e.g. chemical or mechanical trauma) to vessel linings, such as post-reperfusion injury. In addition treatment of arterial thrombosis with various clot lysing agents, such as tissue plasminogen activator (tPA), is often associated with vascular tissue damage.

Administration of the composition by the methods of the invention, alone or in combination with other compositions, for example that may be administered passively, are useful in various clinical situations. These include but are not limited to: 1) acute arterial thrombotic occlusion including coronary, cerebral or peripheral arteries; 2) acute thrombotic occlusion or restenosis after angioplasty; 3) reocclusion or restenosis after thrombolytic therapy (e.g., in an ishemic tissue); 4) vascular graft occlusion; 5) hemodialysis; 6) cardiopulmonary bypass surgery; 7) left ventricular cardiac assist device; 8) total artificial heart and left ventricular assist devices; 9) septic shock; and 10) other arterial thromboses (e.g., thrombosis or thromboembolism where current therapeutic measures are either contraindicated or not effective).

The various methods of the invention are also useful for the treatment of microbial infections. Many microbes, such as bacteria, rickettsia, various parasites, and viruses, bind to vascular endothelium and leukocytes. Thus, the methods of the invention can be used to administer a composition to a patient to prevent binding of a microbe which uses a particular receptor (e.g., selectin) as its binding target molecule, thereby modulating the course of the microbial infection.

The methods of the invention can be used to treat vasculitis by administering to a patient a composition described above. Tissue damage associated with focal adhesion of leukocytes to the endothelial lining of blood vessels is inhibited by blocking the P- and L-selectin receptors, for example.

The dosage ranges for the administration of the compositions in the methods of the invention are those containg a dose of the active agent effective large enough to produce the desired effect in which the symptoms of the disease/injury are ameliorated. The dosage should not be so large as to cause adverse side effects. Generally, the dosage will vary with the age, condition, sex and extent of the disease in the patient and can be determined by one of skill in the art. The dosage can be adjusted by the individual physician in the event of any complication. When used for the treatment of inflammation, post-reperfusion injury, microbial/viral infection, or vasculitis, or inhibition of the metastatic spread of tumor cells, for example, the therapeutic composition may be administered at a dosage which can vary from about 1 mg/kg to about 1000 mg/kg, preferably about 1 mg/kg to about 50 mg/kg, in one or more dose administrations. When the composition contains heparin for local inhibition of hyperplastic intimal growth, the heparin is administered at a dosage which can very from about 50 to about 1,000 IU per kg of body weight. Care should be taken, however, to avoid overdosage, which would adversely affect normal blood clot formation.

Controlled delivery may be achieved by selecting appropriate macromolecules, for example, polyesters, polyamino acids, polyvinyl pyrrolidone, ethylenevinylacetate, methylcellulose, carboxymethylcellulose, protamine sulfate, or lactide/glycolide copolymers. The rate of release of the therapeutic composition may be controlled by altering the concentration of the macromolecule.

Another method for controlling the duration of action comprises incorporating the composition into particles of a polymeric substance such as polyesters, polyamino acids, hydrogels, polylactide/glycolide copolymers, or ethylenevinylacetate copolymers. Alternatively, it is possible to entrap the composition in microcapsules prepared, for example, by coacervation techniques or by interfacial polymerization, for example, by the use of hydroxymethylcellulose or gelatin-microcapsules or poly(methylmethacrolate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

The various parameters including electric field strengths required for the electroporation of any known cell is generally available from the many research papers reporting on the subject, as well as from a database maintained by Genetronics, Inc., San Diego, Calif., assignee of the subject application. The electric fields needed for in vivo cell electroporation are similar in amplitude to the fields required for cells in vitro. These are in the range of from 100 V/cm to several kV/cm. This has been verified by the inventors own experiments and those of others reported in scientific publications.

Pulse generators for carrying out the procedures described herein are and have been available on the market for a number of years. One suitable signal generator is the ELECTRO CELL MANIPULATOR Model ECM 600 commercially available from BTX, a division of Genetronics, Inc., of San Diego, Calif., U.S.A. The ECM 600 signal generator generates a pulse from the complete discharge of a capacitor which results in an exponentially decaying waveform. The electric signal generated by this signal generator is characterized by a fast rise time and an exponential tail. In the ECM 600 signal generator, the electroporation pulse length is set by selecting one often timing resistors marked R1 through R10. They are active in both High Voltage Mode (HVM) (capacitance fixed at fifty microfarads) and Low Voltage Mode (LVM) (with a capacitance range from 25 to 3,175 microfarads).

The application of an electrical field across the cell membrane results in the creation of transient pores which are critical to the eletroporation process. The ECM 600 signal generator provides the voltage (in kV) that travels across the gap (in cm) between the electrodes. This potential difference defines what is called the electric field strength where E equals kV/cm. Each cell has its own critical field strength for optimum electroporation. This is due to cell size, membrane make-up and individual characteristics of the cell wall itself. For example, mammalian cells typically require between 0.5 and 5.0 kV/cm before cell death and/or electroporation occurs. Generally, the required field strength varies inversely with the size of the cell.

The ECM 600 signal generator has a control knob that permits the adjustment of the amplitude of the set charging voltage applied to the internal capacitors from 50 to 500 volts in LVM and from 0.05 to 2.5 kV in the HVM. The maximum amplitude of the electrical signal is shown on a display incorporated into the ECM 600 signal generator. This device further includes a plurality of push button switches for controlling pulse length, in the LVM mode, by a simultaneous combination of resistors parallel to the output and a bank of seven selectable additive capacitors.

The ECM 600 signal generator also includes a single automatic charge and pulse push button. This button may be depressed to initiate both charging of the internal capacitors to the set voltage and to deliver a pulse to the outside electrodes in an automatic cycle that takes less than five seconds. The manual button may be sequentially pressed to repeatedly apply the predetermined electric field.

The waveforms of the voltage pulse provided by the generator in the power pack can be an exponentially decaying pulse, a square pulse, a unipolar oscillating pulse train or a bipolar oscillating pulse train, for example. Preferably, the waveform used for the methods of the invention is an exponential pulse. The voltage applied between the at least first and second electrode is sufficient to cause electroporation of the vessel such the composition delivered to the vessel is retained for a period of time, as described above. The field strength is calculated by dividing the voltage by the distance (calculated for 1 cm separation; expressed in cm) between the electrodes. For example, if the voltage is 500 V between two electrode faces which is ½ cm apart, then the field strength is 500/(½) or 1000 V/cm or 1 kV/cm. Preferably, the amount of voltage applied between the electrodes is in the range of about 10 volts to 200 volts, and preferably from about 50 to 100 volts.

The pulse length can be 100 microseconds (.mu.s) to 100 millisecond (ms) and preferably from about 500 μsec to 10 msec. There can be from about 1 to 10 pulses applied to an area or group of cells. The waveform, electric field strength and pulse duration are dependent upon the exact construction of the catheter device and types of molecules in the composition to be transferred to the cells or vessel via electroporation. One of skill in the art would readily be able to determine the appropriate pulse length and number of pulses.

As applied to delivery of heparin for inhibition of hyperplastic intimal growth in a blood vessel, the term “substantially contemporaneously” means that the electric pulse and the heparin are applied intravascularly (i.e. locally) reasonably close together in time. Preferably, the heparin is administered prior to or concurrently with electropulsing. When applying multiple electrical impulses, the heparin can be administered before or after each of the pulses, at any time between the electrical pulses, or continuously while the pulses continue. Since the heparin is released directly into the vascular region to be treated (e.g., a lesioned arterial segment), administration of the heparin must be sufficiently close in time to administration of the electrical pulses so that blood flow does not carry the heparin away from the treatment site before the electrical pulses cause electroporation of the cells lining the vasculature. Thus, it is recommended that the heparin be released over a sustained interval, for example from about 10 to about 30 seconds, or 15 to about 25 seconds, with the electrical pulses being delivered periodically during at least a portion of the interval of heparin release. The electrical pulses may also continue for a period of several seconds, such as about 5 to 60 seconds following completion of heparin release, depending upon such factors, for example, as the rate and volume of blood flow in the vascular region under treatment, the nature of the tissue to be electroporated, the condition of the patient, and the molecular weight and chemical characteristics of the heparin or derivative used as the therapeutic agent.

Results of tests of the effect of heparin, delivered locally by electroporation, on neointimal growth and vascular remodeling following balloon injury of rat carotid arteries demonstrate the potential value of electroporation technology as an encouraging new drug or gene delivery approach in the area of vascular pathology, such as arterial restenosis. To study the beneficial effect of using electroporation technology in intimal hyperplasia control, local interstitial and intracellular delivery of heparin was used to target a local condition in conjunction with local electroporation of the arterial wall. The animal model chosen (Example 4) was that of balloon injury of a normal artery and not of an atherosclerotic lesion. In this model, highly significant results are evident in that heparin-treated, electroporated vessels consistently show vastly reduced neointimal thickness compared to the heparin-treated, non-pulsed vessels.

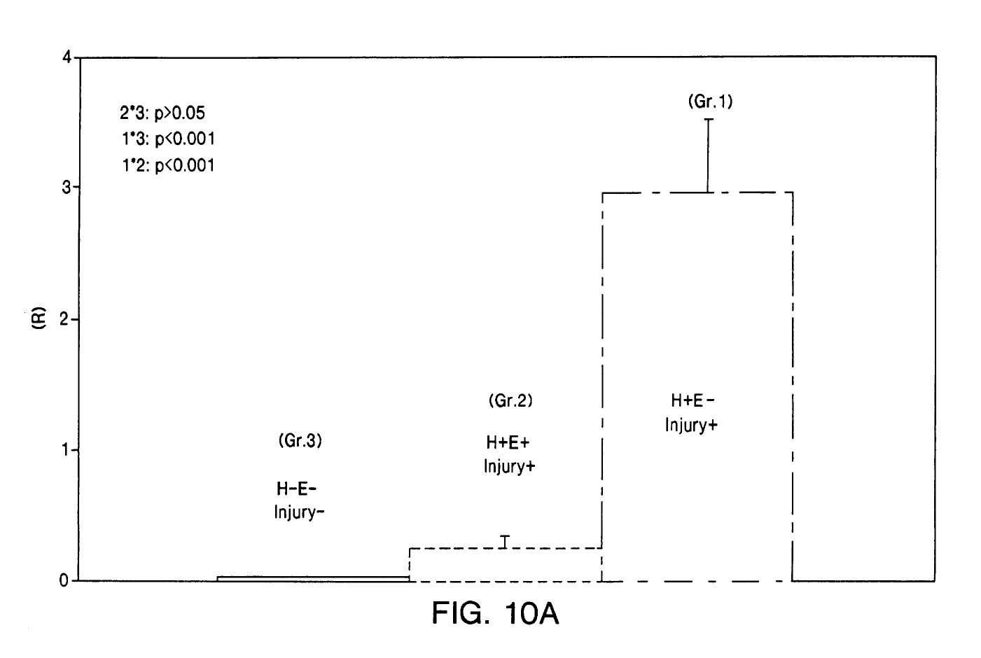

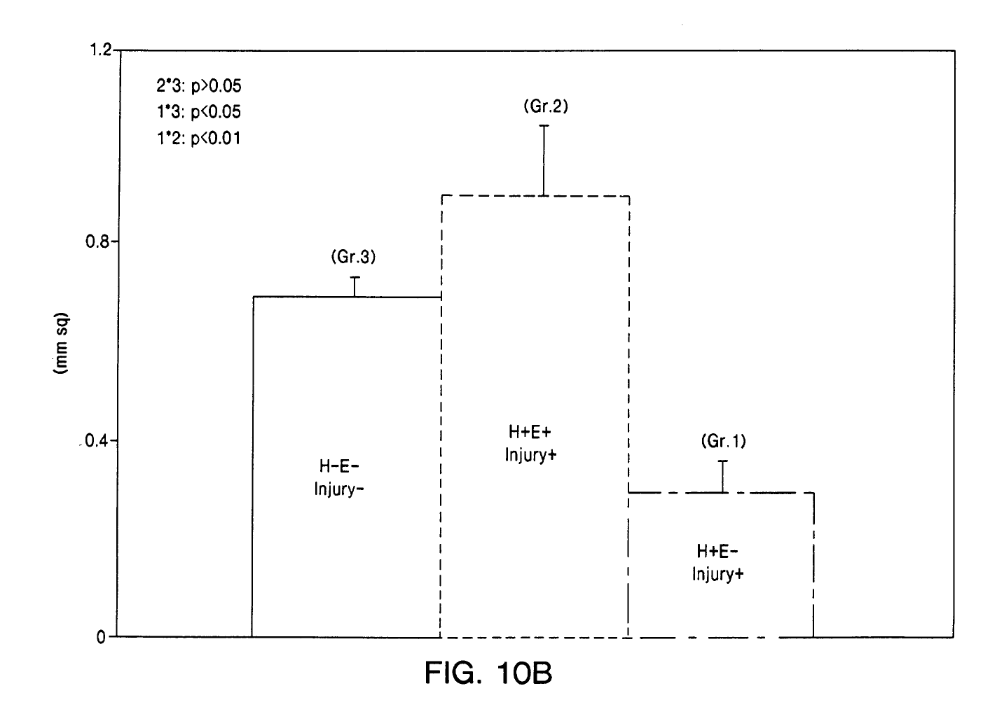

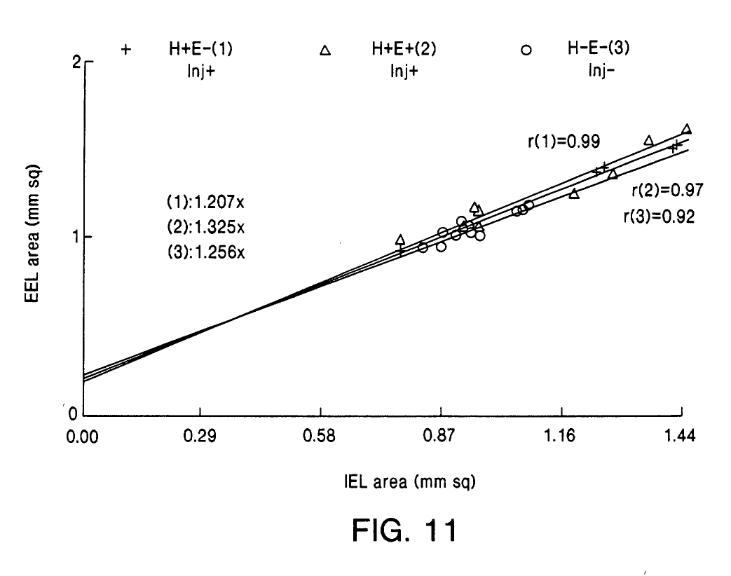

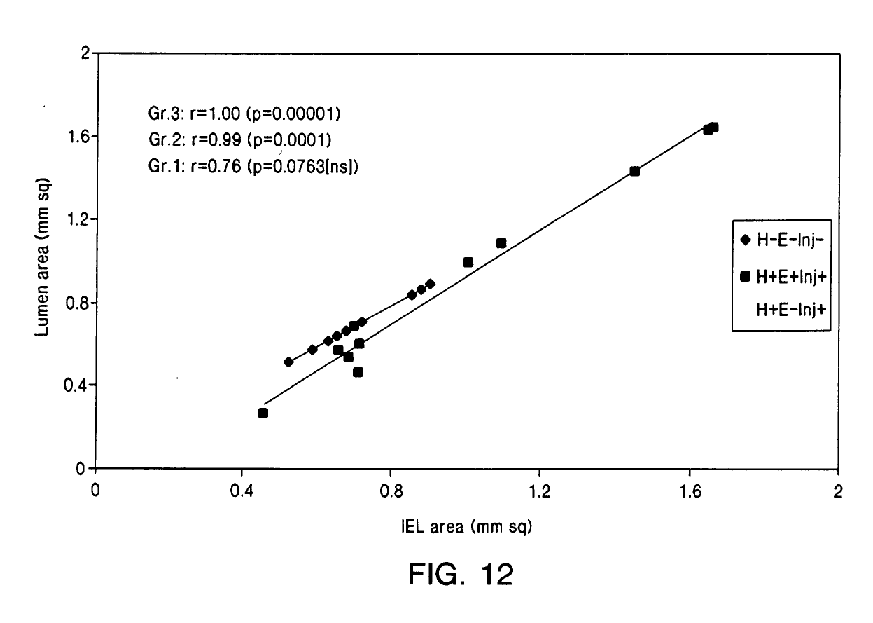

The experimental balloon injury in heparinized but non-electroporated (H+E−) arteries elicited pronounced neointima formation, whereas the electroporated arteries showed strong reduction in the formation of neointimal volume (FIG. 10). Arterial morphometry data of all three experimental populations are presented in Table 1 and graphical representation of the morphometric data is shown in FIGS. 11(A) and 11(B). An excellent correlation was found between the internal and external elastic lamina areas across the groups and this correlation provides evidence of minimal disruption of the morphological relationship by pulsed electric fields (FIG. 12).

The results of this study provide indirect evidence that heparin remains intact and/or biologically active under electroporation. That a compound may retain its chemical and biological activities after electroporation has also been shown by the elimination or dramatic reduction of tumors in animal and clinical studies with intratumoral or intravenous injection of bleomycin followed by electroporation (L. M. Mir Bull Cancer 81(9 :240-248, 1994; G. A. Hofmann et al. supra).

It is known that the vessel diameter enlarges near lesions to accommodate neointimal growth up to a physiological limit. Consequently, vessel dilation partially compensates for the loss of luminal area due to formation of neointima. Experimental studies with stents seem to point to the possibility that most clinical stenosis is a result of failure of vascular remodeling (G. S. Mintz et al., Circulation 94:35-43 (1996)).

However, neointimal growth plays an active part in the stenotic process. The results of the studies described herein show that the relationship of vessel diameter to neointima thickness is not characterized by a monotonic function and in most cases showed a bell shaped configuration. On the other hand, linear regression analysis of data showing the relationship of vessel diameter to neointima thickness showed that the active lumen area correlated well with the IEL area in all three groups, although to a slightly lesser extent in the H+E− group (r−0.76, p=0.07; FIG. 12). The slope for H+E+ (0.57) was steeper than the slope for the H+E− group (0.17), suggesting that vessel wall adaptation (remodeling) to neointimal growth might be more favorable under H+E+ conditions, although the magnitude of this effect would still be insufficient to produce aneurysmal dilatation (M. E. Staab et al. Int J Cardiol 58:31-40, 1997).

The results of this study show inhibition of neointimal growth and favorable remodeling of arterial walls for a period of at least 28 days in rats treated with electroporation-enhanced delivery of heparin to arterial walls.

While the mechanism by which the electroporation-enhanced delivery of heparin, or a derivative thereof, inhibits formation of neointima is not known and does not form a part of this invention, the following possibilities may influence the effect. The antiproliferative and antimigrative effect of heparin on smooth muscle cells as well as the ability of heparin to facilitate reendothelialization of a denuded arterial segment are potential contributors to the observed inhibition of hyperplastic intimal growth in arterial walls treated according to invention methods. Reendothelialization would limit exposure of the underlying smooth muscle cells to plasma and platelet-derived growth factors and other chemotactic substances. It is also possible that electroporation-enhanced delivery of heparin to arterial walls inhibits the deposition of extracellular matrix, an important aspect of arterial response to injury (A. Chajara et al. J Cardiovasc Pharmacol 23:995-1003 (1994)). Furthermore, electroporated heparin may reduce thrombin activity at the site of injury and prevent platelet deposition by blocking the platelet Gplb binding site on von Willebrandt factor (M. Sobel et al. J Clin Invest 87(5):1787-1793, 1991; D. Meyer and J. P. Girma Thromb Haemost 70(1):99-104, 1993). It is also plausible that gene regulatory events, and other biological responses triggered by electroporative delivery of heparin during the very early phases after vessel injury, could switch a mechanism that would interrupt the cascade of reactions leading to restenosis and suppress any subsequent forward events. Heparin is known to inhibit the expression of a variety of genes (e.g., ICAM-1, protein kinase C_{α} and _{δ} (S. J. Miller et al. Thromb Haemost 80(3):481-487, 1998; G. Pintus et al. FEBSL Lett 449(2-3):135-140, 1999) and, since inhibition of gene expression is a relatively rapid phenomenon, short-term down regulation of certain genes may be sufficient to block the initiation of events that ultimately would result in vessel restenosis.

The results of the experiments described herein, particularly in Example 4 below, indicate that the use of electroporation in conjunction with local administration of heparin or other suitable drugs or genes could be a successful prophylactic strategy for injury induced, e.g., angioplasty-induced, restenosis and similar disease conditions, such as intimal thickening which occurs in late saphenous vein bypass graft failure. Moreover, electroporation-enhanced delivery of heparin may have the potential to restrict negative or constrictive remodeling a possible cause of vascular stenosis caused by a variety of disease and traumatic conditions.

The following examples are intended to illustrate but not limit the invention. While they are typical of those that might be used, other procedures known to those skilled in the art may alternatively be used.

### EXAMPLE 1

### Endoluminal Injection of Fluoresceinated Heparin and Pulsed Electrical Stimulation of the Carotid Artery in a Spontaneously Breathing Rabbit

1. Methods

Experiments were performed in 12 New Zealand white rabbits of either sex (2.5-3.4 kg) preanesthetized with xylazine (2 mg.kg⁻¹) and ketamine (50 mg.kg⁻¹) intramuscularly and an injection of alphachloralose (30 mg.kg⁻¹) intraveneously through an ear vein. A supplemental dose of 10 mg.kg⁻¹ chloralose was given every hour. The anesthetic state was maintained such that the toe-pinching reflex and corneal reflexes were absent.

All experiments were conducted in accordance with the guidelines adopted by American Physiological Society on the use of animals for research.

Animals were placed supine and strapped on the surgical table. The trachea was intubated to allow spontaneous breathing of ambient air. Electrocardiogram (EKG) of the animal was obtained by using Lead II in differential mode. End-tidal CO₂ tension was monitored by a CO₂ analyzer (Datex, Puritan-Bennett). Body temperature was kept at the 38-38.5° C. range by radiant heating.

2. Surgical Preparation and Experimental Protocol

A longitudinal incision in the cervical region was made in the rabbit to expose the common carotid arteries on both sides. Approximately 6 cm in length of carotid artery on each side was isolated from the surrounding tissue and vagosympathetic nerve trunk. The caudal end of the carotid artery on one side was transiently occluded with a vascular clip at the junction between the neck and chest. A small incision was then made at the rostral end of the artery just below transversus vein) to push an electroporator catheter (FIG. 1) through this incision. After insertion of the catheter, the catheter balloon was repeatedly inflated for 30 seconds inside the arterial lumen in order to denude the endothelial lining. An indelible ink mark was placed on the inflated portion of the artery. The balloon was then deflated and the catheter tip was held just above the vascular clip.

A 0.2 ml of freshly prepared diluted heparin (1 mg. of fluoresceinated heparin (F-heparin) with an activity of 167 unit/mg (Molecular Probe, Inc.) dissolved in 4 ml) was injected through the one port of a double lumen catheter over a period of about 10 seconds. The catheter was then pulled out of the artery and the vascular clip was taken off from the caudal end to restore blood flow in the artery. Exactly the same procedure was adopted for the contralateral carotid artery (test artery). The only exception was that for the test artery, the carotid artery was stimulated intraluminally using a platinum or silver electrode. Two platinum or silver wires were coiled around the catheter just above the balloon for a length of about 10 mm with an interelectrode distance of 2 mm-3 mm.

Lead II EKG was differentially amplified and the output was continuously monitored on an osciloscope (Tektronix) and recorded on a Gould TA-2000 thermal-array recorder for evaluation. 1-12 hours after heparin injection, both carotid arteries were excised and immediately flash frozen in isopentane pre-chilled in liquid nitrogen. Arteries were stored in −70° C. until further processing.

Arterial segments were subsequently freeze sectioned (10 micron) transversely. Microscopic slides containing arterial sections were observed under a Zeiss confocal laser (argon-krypton) scan microscope (LSM 410 Invert), (excitation at 495 nm and emission at 515 nm) to obtain video image (magnification 40 times) of fluorescence. Subsequently, control and test samples were compared by analyzing fluorescence intensity by Line Intensity Scan at different depths of the arterial wall using commercially obtained software (Image 1:Universal Imaging Corp.).

3. Protocols of Pulsed Stimulation

The luminal wall of the carotid artery was stimulated through bipolar platinum or silver electrodes, which were laid against the luminal surface sufficiently without damage. Pulsed activation of the luminal surface was obtained using an exponential pulse generator (Model ECM 600, BTX, a division of Genetronics, Inc., San Diego, Calif.). Four pulses of 50-60 V amplitude with a pulse width of about 0.5-10 msec (milliseconds) were applied over a period of 60 seconds. This protocol was adopted either for the left or right carotid artery.

4. Observation and Data Analysis

During pulse stimulation of the carotid artery, mild twitching of the cervical region could be seen, but no appreciable change was observed in EKG dynamics over the entire experimental duration.

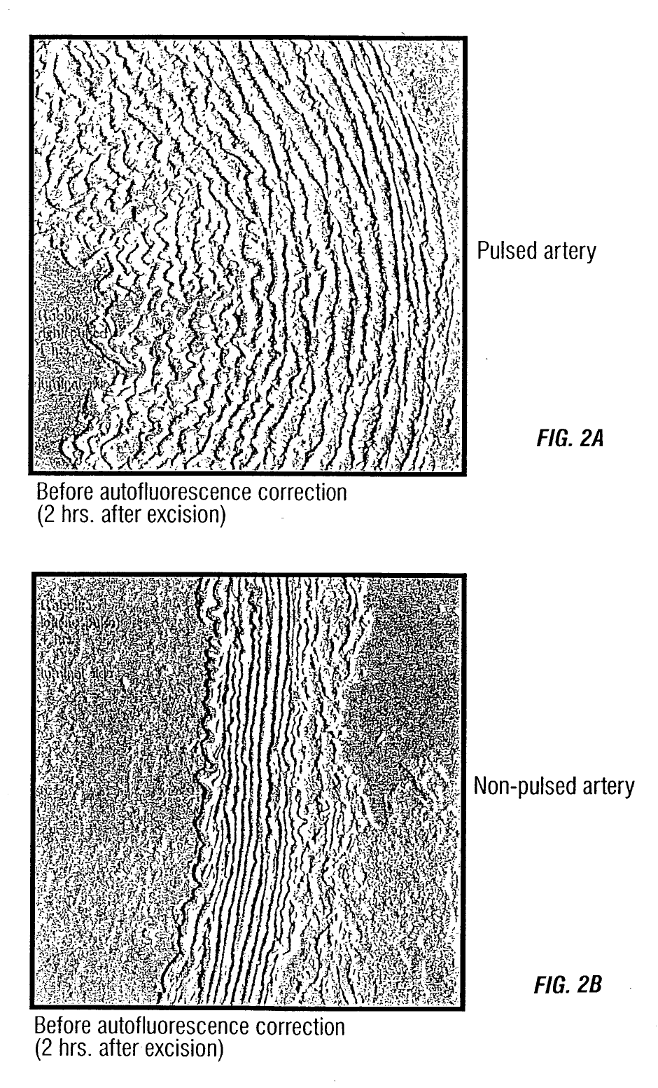

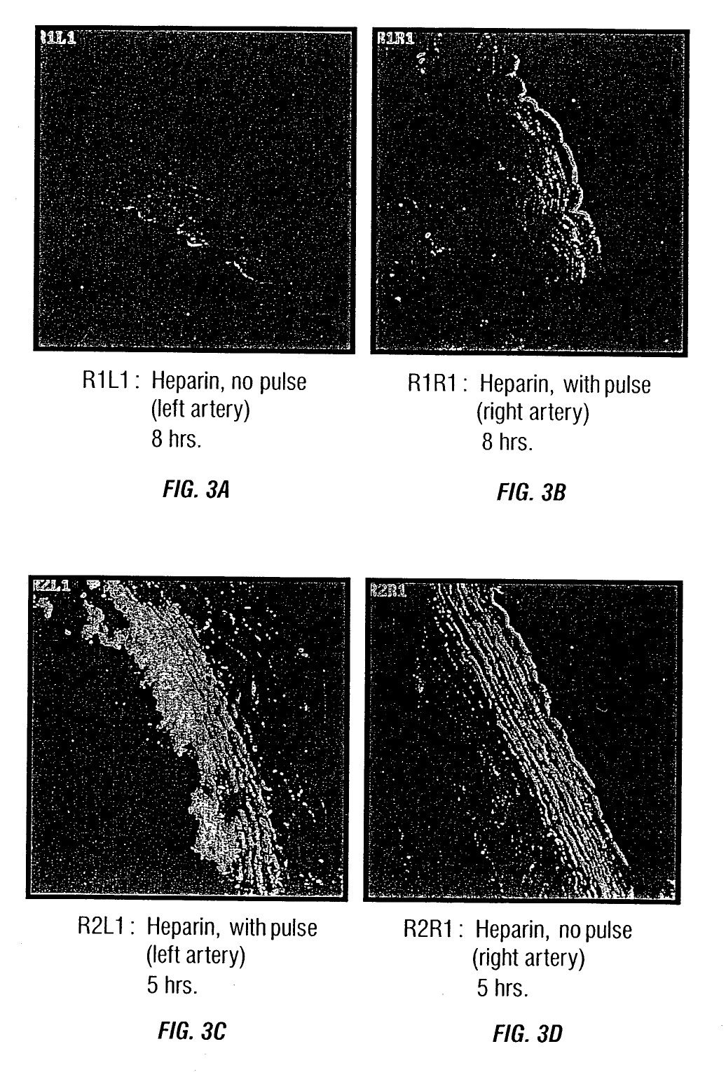

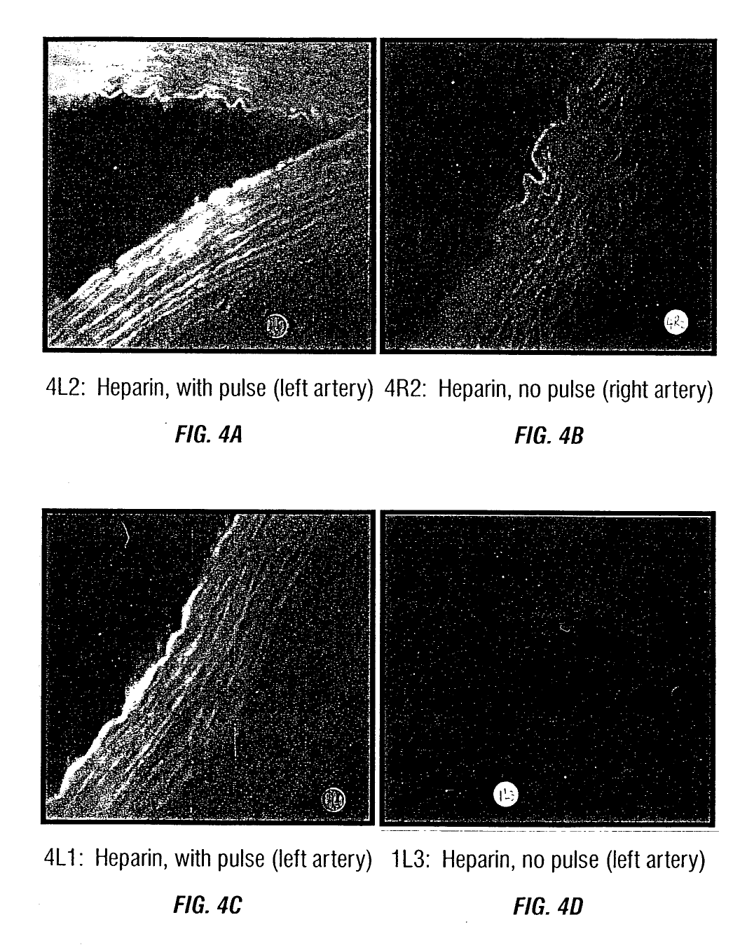

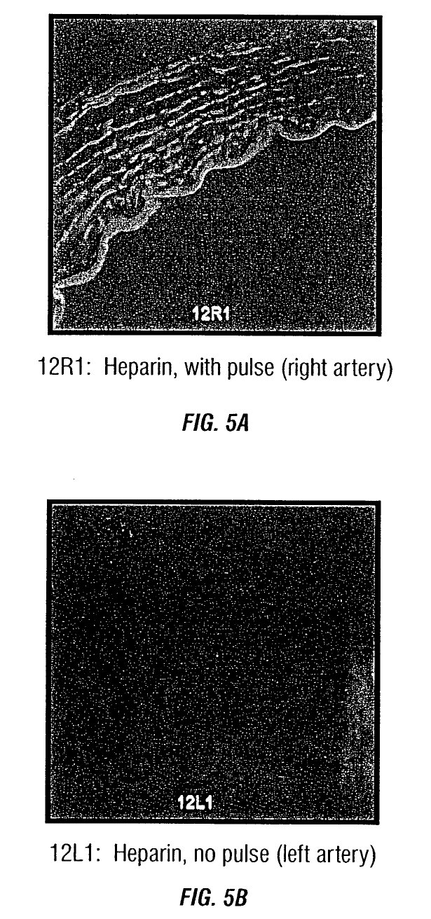

Green fluorescence heparin of the arterial wall could be distinctly seen in the microscopic slide preparations (in different layers of the arterial wall). Confocal scan image of the arterial wall showed penetration of F-heparin in both control and test samples. However, it was evident that the flourescent-intensity in the test sample was much stronger and went into the deeper region of the arterial wall (FIGS. 2-5).

The pulsed electrical stimulation facilitated introduction of F-heparin effectively to the deeper region of arterial wall in a physiologically normal experimental animal. Heparin was mostly present in the media but also in the intima of the vessel wall. However, the intensity dropped significantly towards the adventitia. It is possible that only the portion of the electrode making contact with the luminal wall shows more fluorescence than the adjacent space. From the tissue sectioning, it is not possible to say which portion of the tissue sectioning of the luminal wall sample had contact with the electrodes. However, it is possible that if some sections in the test sample show greater penetration and intensity than the others, those sections probably were in contact with the luminal wall. Also, the fluorescent image could not ascertain if balloon inflation of the bilateral arteries had equal degree of endothelial denudation, the variation in which could alter the penetration of F-heparin among the samples.

FIG. 1 shows a schematic of the catheter used in the above examples. One of the problems of working with fluoresceinated heparin is that there is considerable amount of autofluorescence from the collagen and elastin of the tissue sample. In absolute terms of fluorescent intensity, these tend to distort the real pattern of the fluorescence in the vessel wall due to heparin alone. However, in the present examples, in every case, it is clear that the relative fluorescent intensity was always stronger in the treated vessel that was pulsed compared to the non-pulsed artery. All the photographs had identical magnification (40×) and the brightness and contrast were set to the same level for photography (FIGS. 2-5). All epifluorescence images were monitored in Sony videocon monitor attached to a Hamamatsu CCD camera.

However, by processing the samples at higher pH (9.0), it was possible to considerably reduce or even eliminate the interfering autofluorescence. The photos of FIGS. 2-5 indicated that the local delivery of heparin in the vessel completely washes out in two hours, whereas heparin delivery in the pulsed artery was sustained for at least 12 hours.

### EXAMPLE 2

FIG. 6 shows another configuration for a catheter useful in the methods of the invention, whereby conductive silver paint or a similar conductive material is placed around the catheter covering a length of approximately 2.5 cm. This portion of the catheter is attached to a silver wire which, in turn, is connected to one terminal of a generator, e.g., ECM 600 exponential generator (BTX, a division of Genetronics, Inc., San Diego, Calif.). The second electrode is placed externally and is placed on the abdominal muscle, preferably using a gel for better contact (FIG. 6, shaved area). This second electrode, serving as the anode, is in turn connected to the other terminal of the generator.

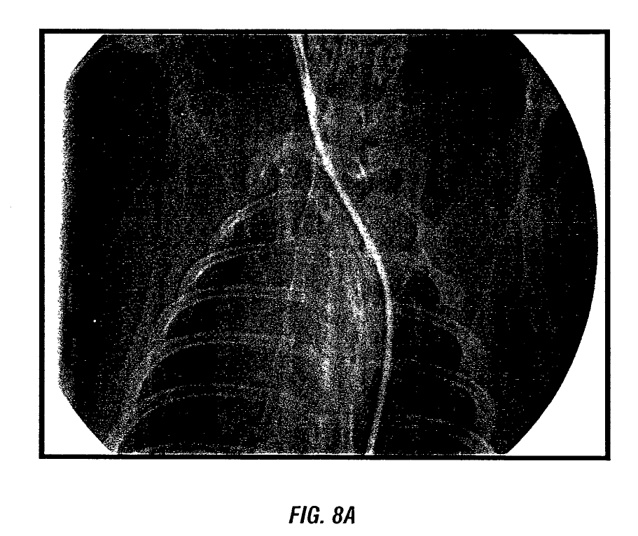

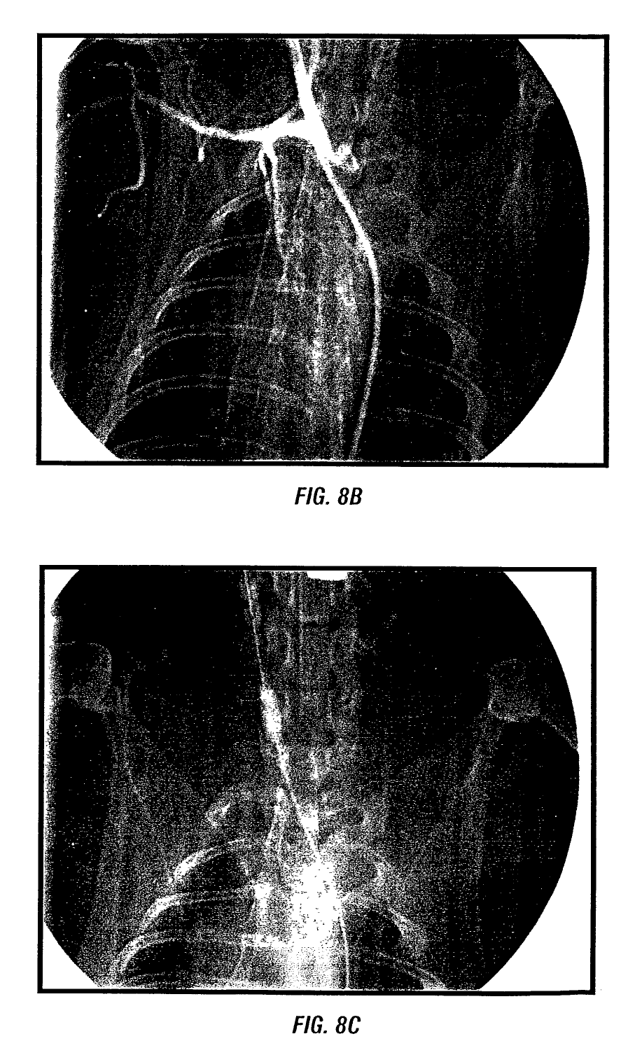

Another embodiment of the catheter comprises one electrode positioned between two balloons and a guidewire acting as a second catheter. Such a configuration is shown in FIG. 7. This catheter was used in the following experiment. Three rabbits weighing about 4 Kg were anesthetized with xylazine (0.1 ml/kg) and ketamine (0.5 ml/kg i.m.). General anesthesia was maintained with α-chloralose (30 mg/Kg. i.v.). Intubation was endotracheal, as described in Example 1. A femoral artery in the leg on one side of the rabbit was exposed. A 5F sheath was introduced and the catheter was pushed under fluoroscopic guidance to the right or left carotid artery. A series of x-rays, FIGS. 8A-C, show successful deployment of the catheter (FIG. A, insertion). Radiocontrast fluid was infused (FIG. B) allowing confirmation of the catheter position, the patient artery, the balloon and the built-in radiopaque marker, as well as presence of the dye in the side branches. After balloon inflation, (FIG. C) 1 ml of fluoresceinated heparin (concentration 1 mg dissolved in 2 ml: biological activity of heparin as per manufacturer: 167 U) was infused between the occluded segment via the drug port and the artery pulsed immediately with the balloons in the inflated condition. Initially, field parameters tested were about 60 V and four pulses each of about 600 μsec pulse length. With these settings, very little uptake of heparin was observed in the treated artery. In a subsequent experiment, voltage and pulse length were changed to 57 V and 22 ms, respectively. As before, four pulses were delivered from ECM 600 pulse exponential generator. The balloon was deflated immediately afterwards with the catheter taken out, but the sheath was left behind to avoid bleeding from the nicked femoral artery. Two hours after infusion of F-heparin, both arteries (treated and the contralateral untreated artery) were taken out for processing. Microscopic images of the treated artery showed massive uptake of the heparin. The fluorescent image of the artery was extremely intense, and the separated arterial sections could not be discerned. Although the control artery also shows fluorescence, visually it was much weaker. Although heparin was not delivered into the control artery, it is obvious that there was systemic circulation from infusion of heparin in the treated artery—part of which must have been taken up by the control artery. In addition, fluorescence due to collagen and elastin was also present. However, both autofluorescence correction at higher pH, as described previously, and computer subtraction of the fluorescence from the control artery from that of the treated artery, showed deep penetration and uptake of the F-heparin in the pulsed artery.

A similar catheter (as depicted in FIG. 7) was also used for a gene marking experiment in a rabbit carotid artery. A New Zealand white rabbit weighing 3.5 Kg was anesthetized with ketamine/xylene cocktail (IM). Intubation was with halothane @1%. After a midline incision, the right common carotid was isolated with silk ligature. 5F sheath was placed into right common carotid over the guidewire after an initial scissor nick in artery. 014″ Schneider guidewire was placed through the sheath into the left iliac artery. The electroporation (EP) catheter was advanced over the wire to left iliac artery. 50% contrast injections with the balloon inflated through the infusion port guided placement to avoid side branches. The infusion sleeve was flushed with saline and the balloons inflated 2 atom. Plasmid (150 μl) (a standard marker gene, lacZ, driven by a CMV promoter) was injected into the infusion port followed by saline. The iliac was pulsed from a BTX ECM 600 exponential pulse generator. Three pulses were given at approximately 10 sec intervals at 76 V and 758 μsec.

For the control artery, balloons were deflated and the wire placed down the right iliac. The procedure was as described above, except that no pulse was applied. The dwell time was about 30 secs. After the procedure, the balloons were deflated and catheters and wires removed. The carotid was ligated proximal and distal to the entry site and the incision was closed in 2 layers. 1500 units of heparin were given after the sheath was in place.

The plasmid DNA was electroporated into the rabbit iliac artery (catheter was guided through to the iliac via the carotid as described above) and gene expression was confined five days later using standard x-gal processing of the artery. In contrast, the control artery did not show detectable gene expression.

### EXAMPLE 3

For further drug delivery studies, the same protocol will be followed as described in detail in Example 1. Forty New Zealand white rabbits will be used for these studies. Time points of approximately 2 hours and 24 hours (group 1) will be tested with balloon catheters as described herein.

Twenty animals, ten animals in each of the time points of group 1, will be used. Both the left and the right arteries will serve as the treated (T) and the control (C). These will be chosen randomly but the number for the T and C will be the same. An ECM 600 pulse generator, which delivers exponential pulses and was used to generate the results described above, will also be used for these experiments.

Ten animals will be tested with square wave pulses from a BTX T820 Square Wave Pulser and arteries will be excised after two hours for subsequent studies. The arteries which will serve as T and C will be randomized. BTX T820 delivers square wave pulses where the number of pulses, the voltage and the pulse length can be adjusted. The voltage is about 60 V and the pulse parameters are: four pulses delivered at 1 Hz each of 40 ms (based on studies with the BTX T820 on rat vascular smooth muscle cell experiments in vitro). Square wave pulses have been known to be gentler to some cells. In this group, there will be five arteries in each of the treated and control category. The inflammatory response of the vessel due to balloon inflation as well as application of the pulsed electric field is also evaluated.

Twenty rabbits will be used where the catheter will be introduced either percutaneously or via a small incision in the femoral. This would give results on twenty treated and twenty control arteries. Arteries will be processed after eight hours. The ECM 600 will be used to deliver exponential pulses. An endoluminal balloon catheter used herein has one electrode between two balloons whereas the guide wire will serve as the second electrode (one design). To facilitate proper viewing of the balloons in the inflated and the deflated position under fluoroscopic guidance, radio-opaque markers will be put in appropriate positions. Calculations suggest that there will be enough field penetration into the arteries to deliver drugs although the electrodes are not in direct contact with the arteries.

For each of the specific aims given above, electric field plots will be generated using a commercially available software package EMP (Field Precision, Albuquerque, N. Mex.). This package solves Poisson's equation is solved numerically by finite elements methods. The initial parameters are electrode geometry, resistivities of the artery from the lumen side and the connective tissue side and the range of field strength to be investigated.

The amount of heparin left in the vessel will be determined in each case following a procedure recommended by Molecular Probe. An InSpeck Microscope Image Intensity Calibration Kit will be used. First, the microscope will be calibrated with the beads (microsphere) provided in the kit and the fluorescein-heparin solution will be equilibrated to the 100% microsphere. Alternatively, for different size microsphere, the available figures for “fluorescein equivalent per microsphere” can be used.

The protocol for reduction of autofluorescence due to collagen and elastin from the arterial wall of the isolated rabbit carotid artery is as follows: Tris-buffered glycerol is prepared (90 ml glycerol and 5 ml of 0.5M Tris-HCl, pH 9.0). This is dispensed in 19 ml aliquots in glass scintillation vials and stored 4° C. 2% n-propyl gallate (npg: anti-fading substance) is prepared in tris-buffer (2 mg npg and 1.0 ml of 0.5M tris-HCl, pH 9.0) is prepared fresh and protected from light. 1 ml of the 2% npg solution is added to 19 ml of tris-buffered glycerol and the solution is protected from light. This is the solution used to mount arterial sections on to the microscopic glass slides. Precaution needs to be taken that the solution is discarded on discoloration. All images will be obtained at 40×magnification under immersion oil (Plan-Neofluor objective). Identical brightness and contrast will be set for all photographs.

### EXAMPLE 4

1. Animals and Surgical Preparation

Mature adult male Sprague-Dawley rats (Harlan, San Diego, Calif.) with an average body weight of 423.8±8.6 (SEM) gm were housed in individual cages in light-dark cycled, temperature-controlled rooms. Standard laboratory chow and water were available ad lib. After a minimum of 7 days acclimatization, the rats were anesthetized with an intramuscular injection of ketamine, 90 mg/kg (Phoenix, St. Joseph, Mo.) and xylazine, 15 mg/kg (Fermenta, Kansas City, Mo.). After anesthesia, they were strapped supine on a surgical table with body temperature maintained at 37° C. using a thermostatic blanket. The animals breathed spontaneously and Lead II electrocardiogram was monitored throughout the experiment. Relevant segments of the displayed EKG signals were stored in a Tektronix 5113 dual beam storage oscilloscope and photographed using a DS-34 Polaroid camera. All procedures conformed to the guiding principles of the American Physiological Society and Institutional Animal Care and Use Committee (Department of Health and Human Services, National Institutes of Health).

2. Arterial Deendothelialization Technique

Neointimal hyperplasia in rats was induced by a technique described earlier by Clowes et al. (A. W. Clowes and M. M. Clowes, supra). Briefly, the left and right common carotid arteries (CCA) were isolated through cervical incision. After isolation of the arteries, the left CCA at the thoracic entry point was temporarily clamped with a bulldog clip. The right CCA served as a control artery and was not surgically manipulated. The left CCA was catheterized through a proximal incision at the junction of the internal and external carotid arteries with a 2F Fogarty embolectomy catheter (Baxter Healthcare Corporation, Irvine, Calif.). The catheter was advanced to the distal CCA, inflated with 0.3 ml of ambient air for 30 sec each time, and retracted to the internal carotid-external carotid junction three times, with one rotation after each passage to ensure uniform injury of the arterial wall. The Fogarty catheter was then withdrawn and exchanged for a porous balloon electroporation (PBE) catheter.

A total of 23 animals were studied. One animal died during the procedure due to ventricular fibrillation (H+E− group). Two animals died suddenly within 1 hour of induction of anesthesia (H−E− group). One animal died on day 10 after the procedure (H+E+ group). Rats tolerated the surgery and arterial electroporation well without any noticeable behavioral or weight change. Immediate pre-sacrificial (post-interventional) recorded weight was 431.5±11.3 gm (not significantly different from pre-intervention recorded weight of 423.7±8.6 gm). Pre-sacrificial (post-interventional) electrocardiogram (Lead II) in all groups did not reveal any rhythm abnormality.

3. Local Drug Delivery Device

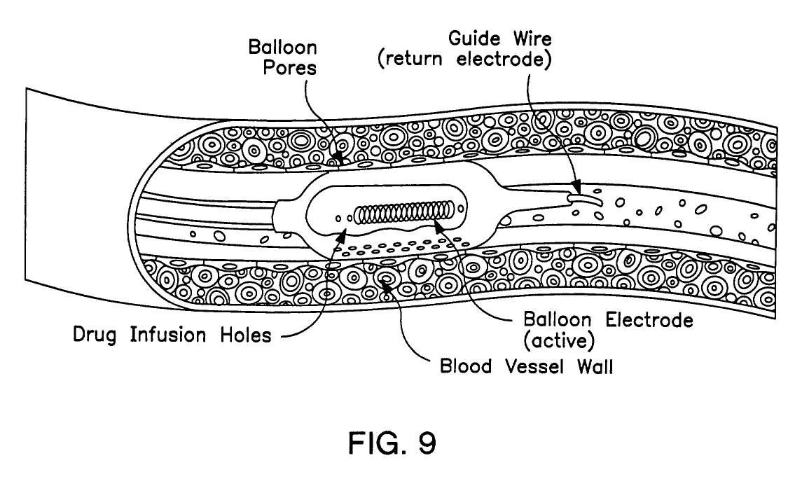

The PBE catheter (manufactured by Danforth Biomedical, Santa Clara, Calif. as per design provided by Genetronics, Inc., San Diego, Calif.) is comprised of a 2F, triple-lumen shaft with a porous 1.5 cm long PET balloon at the distal end. Catheter lumens are used for passage of a 0.014″ guide wire for balloon inflation and deflation and as a conduit for the lead to the central electrode inside the balloon. The balloon has an inflated diameter of 1.4 mm, with four rows of 7 holes each (nominal hole size: 15-25 μm). The balloon serves as a reservoir for the heparin-containing composition. The central electrode inside the balloon is a coiled silver wire wrapped tightly and densely around the catheter shaft, over a length of approximately 10 mm. This balloon electrode acts as the cathode and the inserted guidewire as the anode. The distance between the distal end of the balloon electrode and the proximal end of the bare zone of the conductive guidewire was 4 mm (FIG. 9).

4. Intravascular Local Drug Delivery and Electrical Pulsing Protocol

Following balloon catheter injury of the left CCA, rats were divided into three groups. Groupl received local intracarotid delivery of heparin (Elkins-Sinn, Cherry Hill, N.J.), but no electrical pulsing (positive untreated test: H+E−; H stands for heparin and E for electrical pulsing; + or − designate absence or presence of H or E, respectively). Group 2 received a sequence of four electroporative pulses in the presence of heparin (positive treated test: H+E+). The uninjured right CCA of all animals comprised Group 3 which received neither drug nor electrical pulses and served as negative untreated control (H−E−).

Immediately following balloon injury of the vessel, a PBE catheter was introduced into the injured portion of the left CCA after placement of a temporary clip at the distal end of the balloon injured segment. Heparin (200 IU in 0.1 ml) (Elkins-Sinn) was delivered through the balloon pores into the arterial wall over a period of approx. 20 seconds. Subsequently, within about 10-15 seconds, square-wave electroporative pulses (4 pulses of 100V amplitude and 20 ms pulse duration at 1 Hz) were applied through the catheter using a T-820 Electro Square Porator (BTX, a Division of Genetronics, Inc., San Diego, Calif.). The electrical parameters were chosen based on earlier experiments on enhancement of gene expression by electroporation in skeletal muscle (G. Widera et al., April 1999). After the electroporative pulses, the PBE catheter was withdrawn and the artery was ligated slightly distal to the incision point. Subcutaneous tissue and the cervical skin incision were closed with a layer of 4-0 vicryl suture (Ethicon, Sommerville, N.J.). After surgery, the rats were allowed to recuperate in an environmentally controlled housing facility.

Four weeks after balloon injury and treatment, animals were reanesthetized with ketamine-xylazine and connected to an artificial ventilator (Harvard 683, Nattick, Mass.). Blood vessels were perfused with isotonic saline administered through the left ventricle, followed by pressure fixation at 100 torr with 1% glutaraldehyde in isotonic saline. Carotid arteries (from the proximal edge of the omohyoid muscle to the carotid bifurcation) were removed and placed in 2% glutaraldehyde. After 6 hours, the arteries were segmented in 2-3 mm rings and stored in 70% alcohol until samples were prepared for histology. Tissues were stained either with hematoxylin/eosin or according to Verhoff van Giessen.

5. Histopathology and Quantitative Measurement

All paraffin-embedded stained tissue sections were viewed under a high-resolution light microscope, Olympus BH-2, with attached digital CCD camera, DMC-2 (Polaroid Corp). Images were captured and enhanced in Photoshop environment using a Power PC-9500 and further analyzed by image analysis software (NIH Image V 1.61) available in the public domain. The contour lengths (circumferences, 2πr) were measured at the active lumen-intima boundary (L), the internal elastic lamina (IEL), and the external elastic lamina (EEL). The areas, πr² (r_{L}, r_{IEL} and r_{EEL}) within each circumference were then determined. The area of the intima was then (A_{IEL}-A_{L}) and the area of the media (A_{EEL}-A_{IEL}) (A. W. Clowes et al., Lab Invest 49(2)208-215 (1983)). The intima-to-media ratio (cross-sectional area of the intima over that of the media) was then calculated.

The results of these studies are shown in FIG. 11 herein and Table 1 below.

TABLE 1
Arterial Morphometry at Day 28 After Injury/Treatment

| | Group 1 | Group 2 | Group 3 | | | |
|---|---|---|---|---|---|---|
| | H + E − | H + E + | H − E − | p value | p value | p value |
| | Inj+* | Inj+ | Inj− | 1 Vs 2 | 2 Vs 3 | 1 Vs 3 |
| Intima/media | 2.96 ± | 0.25 ± | 0 | <0.001 | >0.05 | <0.001 |
| thickness ratio | 0.57 | 0.09 | | | | |
| Active lumen | 0.295 ± | 0.902 ± | 0.694 ± | <0.01 | >0.05 | <0.05 |
| area (sq. mm) | 0.06 | 0.14 | 0.041 | | | |
| Medial area | 0.254 ± | 0.338 ± | 0.184 ± | >0.05 | >0.05 | <0.01 |
| (sq. mm) | 0.02 | 0.04 | 0.02 | | | |
| Mean intimal | 0.778 ± | 0.067 ± | 0 | <0.0001 | | |
| area (sq. mm) | 0.16 | 0.02 | | | | |
| Intimal wall | 541 ± | 26.10 ± | 0 | <0.0001 | | |
| thickness (μm) | 87 | 10.00 | | | | |

As compared with a transverse section of uninjured, untreated negative control (H−E− Inj−), 28 days after balloon injury, marked proliferation of neointima was seen in animals which received locally delivered heparin but no electroporation in the balloon injured area (H+E−). By contrast, significant reduction in the volume of neointima was seen in the arteries of animals which received locally delivered heparin in the balloon injured area followed by application of an intravascular electric field. The results of these studies are shown graphically in FIG. 12 (H+E+). H=Heparin, E=Electric pulses. In a large number of histological preparations, the arterial sections were not of typical circular configuration and therefore, the computed area values might be slightly over- or under-estimated.

6. Statistical Evaluation

All values shown in Table 1 and in FIGS. 10-12 are expressed as mean ±SEM. Comparisons between individual groups were performed using either Student-Newmann-Keule for the normally distributed variables or a Mann-Whitney Rank Sum test for the variables that were not normally distributed. Regression lines with adjusted R were calculated by linear regression. A value of p<0.05 was considered significant.

While the invention has been described in detail with reference to certain preferred embodiments thereof, it will be understood that modifications and variations are within the spirit and scope of that which is described and claimed.

What is claimed is:

1. A method for inhibiting or preventing hyperplastic intimal growth in a blood vessel in a subject, said method comprising:
administering a composition comprising heparin or a derivative thereof locally to the vessel in the subject and
applying at least one electric pulse directly to cells lining the vessel, wherein the electric pulse has sufficient strength and duration to cause electroporation of the cells, thereby delivering the composition into the cells so as to prevent or inhibit local hyperplastic intimal growth in the vessel wall as compared with a vessel having non-electroporated cells to which the composition is administered.

2. The method according to claim 1, wherein the vessel wall has been injured.

3. The method according to claim 2, wherein the injury is traumatic.

4. The method according to claim 1, wherein the vessel is an artery.

5. The method according to claim 4, wherein the injury is caused by expansion of the interior diameter of the artery.

6. The method according to claim 5 wherein the expansion was made using a balloon catheter.

7. The method according to claim 1, wherein the hyperplastic intimal growth is restenosis.

8. The method according to claim 1, wherein a plurality of the electric pulses is administered.

9. The method according to claim 7, wherein the plurality comprises at least four pulses.

10. The method according to claim 1, wherein a train of pulses comprising 2 to about 30 pulses is applied.

11. The method according to claim 10 wherein a plurality of the trains is applied.

12. The method according to claim 1 wherein the at least one electrical pulse is monopolar or bipolar.

13. The method according to claim 1 wherein the electric pulse has a voltage from about 50 volts to about 120 volts.

14. The method according to claim 13 wherein the electric pulse has a voltage from about 50 volts to about 90 volts.

15. The method according to claim 1 wherein the pulse duration is in the range from about 100 μsec to about 100 msec.

16. The method according to claim 15 wherein the pulse duration is in the range from about 100 μsec to about 1000 msec.

17. The method according to claim 16 wherein the application of the composition and the administering of the pulse is substantially contemporaneous.

18. The method according to claim 1 wherein the electric pulse is applied via a catheter apparatus.

19. The method according to claim 19 wherein the composition is administered via a catheter apparatus.

20. The method according to claim 18, wherein the composition is contained within a porous balloon portion of the catheter and the method further comprises inflating the balloon of the catheter to contact the interior diameter of the vessel and delivering the composition thereto through pores in the porous balloon.

21. The method according to claim 38 wherein the balloon of the catheter is inflatable to an exterior diameter about 20% in excess of the resting vessel lumen diameter.

22. The method according to claim 1 wherein the electrical pulse is selected from the group consisting of square wave pulses, exponential waves, unipolar oscillating wave forms of limited duration, bipolar oscillating wave forms of limited duration, and other wave forms generating electric fields.

23. The method according to claim 1 wherein the electric pulse has a pulsing frequency of about 1 to 100 Hz.

24. The method according to claim 1 further comprising iontophoresis for delivery of the composition to the cell.

25. The method according to claim 1 wherein the cells are in the adventitial region of the vessel.

26. The method according to claim 21 wherein the derivative is heparin having a molecular weight in the range from about 2500 to about 18,000.

27. A method for electroporation-enhanced local delivery of heparin to cells lining a blood vessel in a subject in need thereof, said method comprising
administering a composition comprising heparin or a derivative thereof locally to the vessel in the subject and
applying at least one electric pulse directly to cells lining the vessel, wherein the electric pulse has sufficient strength and duration to cause electroporation of the cells, thereby delivering the composition into the cells so as to decrease local hyperplastic intimal growth, as compared with an untreated vessel.

28. The method according to claim 27, wherein the vessel has been injured.

29. The method according to claim 28, wherein the injury is traumatic.

30. The method according to claim 29, wherein the vessel is an artery.

31. The method according to claim 28, wherein the vessel is an artery and the injury is caused by expansion of the interior diameter of the artery.

32. The method according to claim 31 wherein the interior diameter of the artery is expanded using a balloon catheter.

33. The method according to claim 27, wherein the hyperplastic intimal growth is restenosis.

34. The method according to claim 27, wherein a plurality of the electric pulses is administered.

35. The method according to claim 27 wherein the at least one electrical pulse is monopolar or bipolar.

36. The method according to claim 27 wherein the electric pulse has a voltage from about 50 volts to about 120 volts.

37. The method according to claim 36 wherein the electric pulse has a voltage from about 50 volts to about 90 volts.

38. The method according to claim 27 wherein the pulse duration is in the range from about 100 μsec to about 100 msec.

39. The method according to claim 27 wherein the pulse duration is in the range from about 100 μsec to about 1000 msec.

40. The method according to claim 27 wherein the application of the composition and the administering of the pulse is substantially contemporaneous.

41. The method according to claim 27 wherein the electric pulse is applied via a catheter apparatus.

42. The method according to claim 41 wherein the composition is administered via a catheter apparatus.

43. The method according to claim 42, wherein the composition is contained within a porous balloon portion of the catheter and the method further comprises inflating the balloon of the catheter to contact the interior diameter of the vessel and delivering the composition thereto through pores in the porous balloon.

44. The method according to claim 43 wherein the balloon of the catheter is inflated to an exterior diameter about 20% in excess of the resting vessel lumen diameter.

45. The method according to claim 27 wherein the electrical pulse is selected from the group consisting of square wave pulses, exponential waves, unipolar oscillating wave forms of limited duration, bipolar oscillating wave forms of limited duration, and other wave forms generating electric fields.

46. The method according to claim 27 wherein the electric pulse has a pulsing frequency of about 1 to 100 Hz.

47. The method according to claim 27 further comprising iontophoresis for delivery of the composition to the cell.

48. The method according to claim 27 wherein the cells are in the adventitial region of the vessel.

49. The method according to claim 27 wherein the derivative is low molecular weight heparin having a molecular weight in the range from about 2,500 to about 18,000.
